(19) United States
(12) Patent Application Publication
(10) Pub. No.: US 2009/0239954 A1
(43) Pub. Date: Sep. 24, 2009

(54) PHOSPHATE BUFFERED OPHTHALMIC SOLUTIONS DISPLAYING IMPROVED EFFICACY

(76) Inventors: Gary L. Collins, Jacksonville, FL (US); Marcie Hargiss, Jacksonville, FL (US); Mark Lada, St. Augustine, FL (US); Robert T. McKee, Jacksonville, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/399,681

(22) Filed: Mar. 6, 2009

Related U.S. Application Data
(60) Provisional application No. 61/037,894, filed on Mar. 19, 2008.

(51) Int. Cl. A61K 8/22; A61P 27/02

(52) U.S. Cl. 514/714

(57) ABSTRACT

The present invention relates to ophthalmic compositions comprising a pH between about 6 and about 8 and about 50 to about 1000 ppm hydrogen peroxide and at least one phosphate buffer. The ophthalmic compositions of the present invention display improved antifungal efficacy against fungi, including candidas albicans and fusarium solani.

### RELATED APPLICATIONS

This application claims priority from U.S. provisional application Ser. No. 61/037,894, which is incorporated by reference in its entirety.

### BACKGROUND OF THE INVENTION

There are many commercially available ophthalmic solutions. Solutions which are used for the cleaning and care of ophthalmic devices, such as contact lenses, should provide disinfection against a variety of bacteria and fungi, which can come in contact with the eye and devices which reside on the eye, such as contact lenses. The solutions must remain free from contamination during the use life of the solution. To meet this requirement solutions either contain a preservative component or are sterile packaged in single use dosages. For contact lens cleaning and care solutions, and over the counter eye drops, multidose containers are popular. These solutions require the inclusion of preservatives (for eye drops) and disinfecting compositions (for contact lens cleaning and care solutions).

Hydrogen peroxide has been used as disinfectant or preservative in ophthalmic solutions. However, hydrogen peroxide is not stable, and must either be included in concentrations which sting the eye or the solutions must contain additional components to stabilize the hydrogen peroxide. Compounds disclosed to be useful as peroxide stabilizers include phosphonates, phosphates, and stannates, and specific examples physiologically compatible salts of phosphonic acids such as diethylenetriamine pentamethylenephosphonic acid. Amino polycarboxylic acid chelating agents, such as ethylene diamine tetraacetic acid have also been disclosed.

Diethylenetriamine pentamethylenephosphonic acid (PTPPA) and ethylenediamine tetraacetic acid (EDTA) are cyctotoxic at relatively low levels and have low pH. Thus, these stabilizers can be included only in small amounts, and require the addition of neutralizing agents to provide a solution that is compatible with the human eye.

Many of these solutions are buffered to help maintain a desired pH throughout storage and usage. Many buffering solutions are known to be useful in ophthalmic solutions.

Accordingly, for solutions that are instilled directly in the eye, or for contact lens cleaning and care solutions that do not need to be rinsed off before the lens is placed on the eye, additional hydrogen peroxide stabilizers are desired.

### SUMMARY OF THE INVENTION

The present invention relates to ophthalmic compositions comprising a pH between about 6 and about 8, at least one phosphate buffer and about 50 to about 1000 ppm hydrogen peroxide. In another embodiment, the present invention relates to ophthalmic compositions comprising a pH between about 6 and about 8, at least one phosphate buffer, about 50 to about 1,000 ppm hydrogen peroxide and between about 50 ppm to about 1,500 ppm of at least one salt of diethylenetriamine pentaacetic acid.

In another embodiment, the present invention relates to ophthalmic compositions comprising a pH between about 6 and about 8, at least one phosphate buffer, about 50 to about 1,000 ppm hydrogen peroxide and about 100 ppm to about 2000 ppm of at least one chlorite compound. In yet another embodiment, the present invention relates to ophthalmic compositions comprising a pH between about 6 and about 8, at least one phosphate buffer, about 50 to about 1,000 ppm hydrogen peroxide and about 100 ppm to about 2,000 ppm of at least one chlorite compound and about 20 to 100 ppm of at least one saturated, polymeric quaternium salt.

The present invention further relates to ophthalmic solutions comprising the components listed in Table 1, in the amounts listed in Table 1.

### DESCRIPTION OF THE INVENTION

The present invention relates to novel ophthalmic solutions comprising low concentrations of hydrogen peroxide and a phosphate buffer. The present invention further relates to ophthalmic solutions comprising small concentrations of hydrogen peroxide which are storage stable and efficacious against bacteria and fungi.

As used herein storage stable, means that under storage conditions, such as temperatures of less than about 40° C., the solution loses less than thirty percent of the hydrogen peroxide in said solution over thirty days, and in some embodiments less than about 25% in thirty days.

Ophthalmic compositions are any composition which can be directly instilled into an eye, or which can be used to soak, clean, rinse, store or treat any ophthalmic device which can be used placed in or on the eye. Examples of ophthalmic compositions include ophthalmic device packing solutions, cleaning solutions, conditioning solutions, storage solutions, eye drops, eye washes, as well as ophthalmic suspensions, gels and ointments and the like. In one embodiment of the present invention, the ophthalmic composition is an ophthalmic solution.

Ophthalmic devices include any devices that can be placed on the eye, or any part of the eye, such as, but not limited to under the eyelid or in the punctum. Examples of ophthalmic devices include contact lenses, ophthalmic bandages, ophthalmic inserts, punctal plugs and the like.

The ophthalmic compositions of the present invention comprise between about 50 to about 1000 ppm hydrogen peroxide. In some embodiments the hydrogen peroxide is present in concentrations between about 100 and about 500 ppm, and in other embodiments, between about 100 and about 300 ppm.

Alternatively, the composition may include a source of hydrogen peroxide. Suitable hydrogen peroxide sources are known, and include peroxy compounds which are hydrolyzed in water. Examples of hydrogen peroxide sources include alkali metal perborates or percarbonates such as sodium perborate and sodium percarbonate.

The compositions of the present invention also comprise at least one phosphate buffer. Suitable phosphate buffers are derived from phosphoric acid, and a base selected from KOH, NaOH, or the potassium or sodium salts of phosphoric acid, and mixtures thereof and the like. Suitable salts include sodium phosphate dibasic and monobasic, potassium phosphate monobasic and mixtures thereof. Total phosphate concentrations for the buffer solution include about 5 to about 100 mmol and in some embodiments between about 25 to about 50 mmol. The concentrations of the phosphoric acid and base or salt may be varied to achieve the desired pH. Solutions of the present invention have a pH of about 6 to about 8, and preferably about 7. For these solutions the phosphate buffer solutions comprising from about 0.05 wt % to about 0.4 wt % monobasic phosphoric acid salt and from about 0. 1 wt % to about 0.8 wt % dibasic phosphoric salt. The total phosphate buffer is present in the ophthalmic composition of the present invention in amounts between about 0.15 to about 1 weight %, based upon the weight of all components in the composition, including water.

It has been surprisingly found that when ophthalmic solutions comprising hydrogen peroxide are formulated using a phosphate buffer instead of a borate buffer, the resulting solution displays unexpectedly improved efficacy against fungi, such as candidas albicans and fusarium solani. This was particularly surprising as little initial improvement in chemical stability of the phosphate buffered solutions was found compared to borate buffered solutions having the same components. Also, borate buffered solutions are conventionally considered bacteriostatic, whereas phosphates have been considered to be nutrients for microbes. The present invention has shown just the opposite effect.

The ophthalmic composition comprising hydrogen peroxide in the amounts described above may be stabilized by including between about 0.005 wt % (50 ppm) to about 0.15 wt % (1,500 ppm), and in some embodiments from about 100 to about 1000 ppm of at least one ophthalmically compatible stabilizer, such as at least one salt of diethylenetriamine pentaacetic acid comprising at least one ophthalmically compatible salt which is soluble in the phosphate buffer. Suitable salts include monocalcium salt or zinc salt of diethylenetriamine pentaacetic acid. Examples include monocalcium salts of DTPA, monozinc salts of DTPA, mixtures thereof and the like. The salts of the present invention may further comprise any additional ophthalmically compatible cations such as sodium, magnesium, combinations thereof and the like, so long as said salts are soluble in the phosphate buffer. In one embodiment the DTPA salt comprises monocalcium DTPA. The concentration of the diethylenetriamine pentaacetic acid salt is between about 50 and about 1000 ppm.

The DTPA salts may formed separately and added to the solution or pentetic acid (diethylenetriamine pentaacetic acid) and a hydroxide salt of the desired cation may be added to the solution in a stoichiometric amount to form the desired DTPA salt in situ.

The ophthalmic compositions of the present invention also have a pH of between about 6 and 8, and in some embodiments between about 6.5 and about 7.5. This allows the compositions of the present invention to be instilled directly in the eye, and to be used on ophthalmic devices that are to be placed in the ocular environment.

The ophthalmic compositions may further comprise at least one additional peroxide stabilizer. Any known peroxide stabilizer may be used, so long as it is not cytotoxic at the concentrations being used, and is compatible with the other ophthalmic composition components. For example, the additional peroxide stabilizer should not interfere with the functioning of any other components included in the composition, and should not react with any other components. Examples of suitable additional peroxide stabilizers include phosphonates, phosphates, ethylene diamine tetraacetic acid, nitrilo triacetic acid, ophthalmically compatible water soluble salts of any of the foregoing, mixtures thereof, and the like. In one embodiment the additional peroxide stabilizer comprises DTPPA or least one pharmaceutically acceptable salt of DTPPA.

The at least one additional peroxide stabilizer may be present in concentrations up to about 1000 ppm, and in some embodiments between about 100 and about 500 ppm. When the additional peroxide stabilizer comprises DTPPA or at least one pharmaceutically acceptable salt of DTPPA, it is present in a concentration up to about 1000 ppm, and in some embodiments between about 100 ppm to about 500 ppm.

The ophthalmic compositions of the present invention may further comprise additional components such as, but not limited to pH adjusting agents, tonicity adjusting agents, buffering agents, active agents, lubricating agents, disinfecting agents, viscosity adjusting agents, surfactants and mixtures thereof. When the ophthalmic composition is an ophthalmic solution, all components in the ophthalmic solution of the present invention should be water soluble. As used herein, water soluble means that the components, either alone or in combination with other components, do not form precipitates or gel particles visible to the human eye at the concentrations selected and across the temperatures and pH regimes common for manufacturing, sterilizing and storing the ophthalmic solution.

The pH of the ophthalmic composition may be adjusted using acids and bases, such as mineral acids, such as, but not limited to hydrochloric acid and bases such as sodium hydroxide.

The tonicity of the ophthalmic composition may be adjusted by including tonicity adjusting agents. In some embodiments it is desirable for the ophthalmic composition to be isotonic, or near isotonic with respect to normal, human tears. Suitable tonicity adjusting agents are known in the art and include alkali metal halides, phosphates, hydrogen phosphate and borates. Specific examples of tonicity adjusting agents include sodium chloride, potassium chloride, calcium chloride, magnesium chloride, zinc chloride, combinations thereof and the like.

The ophthalmic composition may further comprise at least one buffering agent which is compatible with diethylenetriamine pentaacetic acid salt. Examples of suitable buffering agents include borate buffers, phosphate buffers, sulfate buffers, combinations thereof and the like. In one embodiment the buffering agent comprises borate buffer. In another embodiment, the buffering agent comprises phosphate buffer. Specific examples include borate buffered saline and phosphate buffered saline.

The ophthalmic composition may also comprise at least one disinfecting agent in addition to hydrogen peroxide. The disinfecting agent should not cause stinging or damage to the eye at use concentrations and should be inert with respect to the other composition components. Suitable disinfecting components include polymeric biguanides, polymeric quarternary ammonium compounds, chlorites, bisbiguanides, quarternary ammonium compounds and mixtures thereof.

In one embodiment, the disinfecting component comprises at least one chlorite compound. Suitable chlorite compounds include water soluble alkali metal chlorites, water soluble alkaline metal chlorites and mixtures thereof. Specific examples of chlorite compounds include potassium chlorite, sodium chlorite, calcium chlorite, magnesium chlorite and mixtures thereof. In one embodiment the chlorite compound comprises sodium chlorite.

Suitable concentrations for the chlorite compound include concentrations between about 100 and about 2000 ppm, in some embodiments between about 100 and about 1000 ppm, in other embodiments between about 100 and about 500 ppm and in other embodiments between about 200 and about 500 ppm.

Combinations of suitable peroxide/chlorite disinfecting agents are disclosed in U.S. Pat. No. 6,488,965, U.S. Pat. No. 6,592,907, US20060127497, US2004/0037891, US 2007/0104798. These patents as well as all other patent disclosed herein are hereby incorporated by reference in their entirety.

The ophthalmic compositions of the present invention may further comprise at least one additional disinfecting compound selected from the group consisting of fully saturated, polymeric quaternium salts such as poly[oxyethylene(-dimethylimino) ethylene-(dimethylimino)ehthylene dichloride (CAS designation of 31512-74-0, and referred to herein as “Polyquaternium-42”), disclosed in U.S. Pat. No. 5,300,296 and U.S. Pat. No. 5,380,303. The polymeric quaternium salts are desirably fully saturated to insure they are stable in the presence of the hydrogen peroxide. The fully saturated, polymeric quaternium salts may be present in the solution in amounts between about 10 to about 100 ppm and in some embodiments between about 25 to about 100 ppm. It has been found that when at least one fully saturated, polymeric quaternium salts such as Polyquaternium-42 is included in an ophthalmic solution along with hydrogen peroxide and chlorite the resulting solutions display surprisingly improved antifungal properties, particularly against fusarium solani.

One or more lubricating agents may also be included in the ophthalmic composition. Lubricating agents include water soluble cellulosic compounds, hyaluronic acid, and hyaluronic acid derivatives, chitosan, water soluble organic polymers, including water soluble polyurethanes, polyethylene glycols, combinations thereof and the like. Specific examples of suitable lubricating agents include polyvinyl pyrrolidone (“PVP”), hydroxypropyl methyl cellulose, carboxymethyl cellulose, glycerol, propylene glycol, 1,3-propanediol, polyethylene glycols, mixtures there of and the like. Generally lubricating agents have molecular weights in excess of 100,000. When glycerol, propylene glycol and 1,3-propanediol are used as lubricating agents, their molecular weights are lower than 100,000.

When a lubricating agent is used, it may be included in amounts up to about 5 weight %, and in some embodiments between about 100 ppm and about 2 weight %.

One or more active agent may also be incorporated into the ophthalmic solution. A wide variety of therapeutic agents may be used, so long as the selected active agent is inert in the presence of peroxides. Suitable therapeutic agents include those that treat or target any part of the ocular environment, including the anterior and posterior sections of the eye and include pharmaceutical agents, vitamins, nutraceuticals combinations thereof and the like. Suitable classes of active agents include antihistamines, antibiotics, glaucoma medication, carbonic anhydrase inhibitors, anti-viral agents, anti-inflammatory agents, non-steroid anti-inflammatory drugs, antifungal drugs, anesthetic agents, miotics, mydriatics, immunosuppressive agents, antiparasitic drugs, anti-protozoal drugs, combinations thereof and the like. When active agents are included, they are included in an amount sufficient to product the desired therapeutic result (a “therapeutically effective amount”).

The ophthalmic composition of the present invention may also include one or more surfactants or detergents. Suitable examples include tyloxapol, poloxomer (poly(ethylene oxide)-b-poly(propylene oxide)-b-poly(ethylene oxide)) type surfactants which are commercially available from BASF and poloxamine type surfactants (non-ionic, tetrafunctional block copolymers based on ethylene oxide/propylene oxide, terminating in primary hydroxyl groups, commercially available from BASF, under the tradename Tetronic). A specific example is Pluronic F-147 and Tetronic 1304. Surfactants may be used in amounts up to about 5 weight %, and in some embodiments up to about 2 weight %. Tyloxapol is a non-ionic, low molecular weight surfactant, and is fully soluble in the phosphate buffers included in the compositions of the present invention. Tyloxapol is a detergent commercially available from Pressure Chemical Company. In embodiments where tyloxapol is included, it is included in amounts between about 500 to about 2000 ppm.

Some surfactants may also act as disinfectant enhancers. Disinfectant enhancers for the solutions of the present application include C₅₋₂₀ polyols, such as 1,2-octanediol (caprylyl glycol), glycerol monocaprylate, sorbitan monolaurate (TWEEN 80) combinations thereof and the like. Disinfectant enhancers may be present in amounts from about 50 to about 2000 ppm.

The compositions of the present invention may also comprise additional optional components such as chelating agents, demulcents, humectants and the like. Examples include citrates, succinates, cellulosic materials, amphoteric surfactants, non-ionic surfactants, mixtures thereof and the like.

Additionally, the ophthalmic composition may comprise one or more viscosity adjusting agent or thickener. Suitable viscosity adjusting agents are known in the art and include polyvinyl alcohol, polyethylene glycols, guar gum, combinations thereof and the like. The viscosity adjusting agent may be used in amounts necessary to achieve the desired viscosity.

It will be appreciated that all the components at the concentrations they are used herein, will be soluble in buffered solutions, compatible with the other solution components and will not cause ocular pain or damage.

Examples of ophthalmic solutions according to the present invention are disclosed in Table 1.

TABLE 1

| | Chemical | | |
|---|---|---|---|
| Component | Formula | Concentration | |
| Hydrogen Peroxide | H₂O₂ | 50-500 | ppm |
| Sodium Chlorite | NaClO₂ | 100-1000 | ppm |
| Potassium Phosphate, | KH₂PO₄ | 0.15 to 1% | |
| monobasic | | | |
| Sodium Phosphate, | Na₂HPO₄•2H₂O | | |
| Dibasic | | | |
| Polyquaternium-42 | (CH₁₀H₂₄N₂O•2Cl)ₙ | 0-100 | ppm |
| (WSCP, polixetonium) | | 10 to 100 | ppm |
| Polyvinylpyrrolidone | (C₆H₉NO)ₙ | 0-2500 | ppm |
| K90 | | 500 to 2500 | ppm |
| (PVP, Povidone) | | | |
| DTPA, monocalcium salt | CaC₁₄H₂₃N₃O₁₀ | 0 to 1,000 | ppm |
| Poloxamer 407 | OH(C₂H₄O)₁₀₁(C₃H₆)₅₆—(C₂H₄O)₁₀₁H | 500-10,000 | ppm |
| Tyloxapol | (C₁₄H₂₂O•C₂H₄O•CH₂O) | 0-5,000 | ppm |
| | | 250-5,000 | ppm |
| Sodium Citrate | Na₃C₆H₅O₇•2H₂O | 0-6500 | ppm |
| | | 650-6500 | ppm |

| | | |
|---|---|---|
| Sodium Chloride | NaCl | Adjusted to Tonicity |
| Purified Water | H₂0 | Q.S. |

Ophthalmic solutions of the present invention may be formed by mixing the selected components with water. Other ophthalmic compositions may be formed by mixing the selected components with a suitable carrier.

In order to illustrate the invention the following examples are included. These examples do not limit the invention. They are meant only to suggest a method of practicing the invention. Those knowledgeable in contact lenses as well as other specialties may find other methods of practicing the invention. However, those methods are deemed to be within the scope of this invention.

### EXAMPLES

### Examples 1-8

A series of solutions with different buffers (borate buffer and phosphate buffers with high and low buffer concentrations) were made as follows. For each solution, 1.5 gm PVP K90 (ISP) and 4.5 gm poloxamer F-172 (BASF) were weighed into about 100 ml deionized water and gently heated to allow all of the material to dissolve. The PVP solution was allowed to cool and an additional ˜500 ml deionized water was added.

The sodium chloride and buffer components (either boric acid and sodium borate decahydrate or monobasic potassium phosphate and disodium hydrogen phosphate dihydrate) were added to each solution in the amount listed in Table 2. The salt of DTPA (ISP Columbus) listed in Table 2 was added in the amount listed in Table 2. The solution was mixed thoroughly until all components were fully dissolved. The solution was titrated with NaOH solution (0.1N) until the pH was 7.2-7.4.

Deionized water was added to make up a total of approximately 950 ml. The pH was checked and corrected to 7.2-7.4, if necessary. Sodium chlorite (0.63 gm, Acros) and 0.7 gm hydrogen peroxide (30%; Fisher Scientific) were added and mixed thoroughly. The pH was rechecked and neutralized with NaOH solution as necessary and 0.16 gm PQ-42 was added. Deionized water was added to make up to 1000 g total.

The solutions were stored in opaque polypropylene or high density polyethylene containers.

TABLE 2

| | | Wt (gm) | | | | | | | |
|---|---|---|---|---|---|---|---|---|---|
| Component | Source | Ex 1 | Ex 2 | Ex 3 | Ex 4 | Ex 5 | Ex 6 | Ex 7 | Ex 8 |
| H₃BO₃ | Fisher | 4.5 | 4.5 | 4.5 | 4.5 | 0 | 0 | 0 | 0 |
| | Science | | | | | | | | |
| | ED | | | | | | | | |
| Na₂B₄O₇•10H₂O | Fisher | 0.25 | 0.25 | 0.25 | 0.25 | 0 | 0 | 0 | 0 |
| | Science | | | | | | | | |
| | ED | | | | | | | | |
| CaDTPA | ISP | 0.4 | 0 | 0.4 | 0 | 0.4 | 0 | 0.4 | 0 |
| | Columbus | | | | | | | | |
| Na₅DTPA | ISP | 0 | 0.4 | 0 | 0.4 | 0 | 0.4 | 0 | 0.4 |
| | Columbus | | | | | | | | |
| Tyloxapol | Sigma | 0 | 0 | 1 | 1 | 0 | 0 | 1 | 1 |

100 g aliquots of each of the solutions of Examples 1-8 were placed in opaque plastic containers and labeled. A 5 ml sample from each container was removed and analyzed for hydrogen peroxide using the metavanadate calorimetric method, according to the method disclosed in Talanta, vol. 66, issue 1, pg 86-91, Mar. 31, 2005. This is the baseline (t=0) hydrogen peroxide concentration, reported in the fourth column of Table 3, below. Each container was weighed, and the baseline weights were recorded. The containers were stored at 40° C. At each of the intervals shown in Table 3, each container was weighed and 5 ml sample was removed for hydrogen peroxide determination as described above. The results are shown in Table 3. The value for Δppm was calculated by subtracting the concentration hydrogen peroxide in each solution measured at the time shown in Table 4, and subtracting from the original hydrogen peroxide concentration for that sample. The % Δ was calculated by dividing the concentration of hydrogen peroxide in each solution measured at the time shown in Table 3, by the original hydrogen peroxide concentration for that sample.

TABLE 3

| | | DTPA | [Tyloxapol] | [H₂O₂] ppm | | | |
|---|---|---|---|---|---|---|---|
| Ex # | buffer | Type | ppm | initial | 1 wk | 2 wks | 3 wks |
| 1 | Borate | Ca | 0 | 226 | 214 | 190 | 174 |
| 2 | Borate | Na | 0 | 228 | 203 | 168 | 143 |
| 3 | Borate | Ca | 1000 | 226 | 214 | 168 | 166 |
| 4 | Borate | Na | 1000 | 227 | 202 | 159 | 139 |
| 5 | Phosphate | Ca | 0 | 230 | 213 | 194 | 183 |
| 6 | Phosphate | Na | 0 | 229 | 209 | 186 | 185 |
| 7 | Phosphate | Ca | 1000 | 229 | 214 | 197 | 185 |
| 8 | Phosphate | Na | 1000 | 229 | 209 | 185 | 184 |

The phosphate buffered solutions display stability throughout the 3 week test period which is equivalent to the borate buffered solutions or slightly better.

### Examples 8-14

Example 5 was repeated, except that the components listed in Table 4, below were used in the amounts listed in Table 4. All other components used in Example 5 (hydrogen peroxide, sodium chlorite, PVP, Poxamer 147) were used in the amounts specified in Example 5.

TABLE 4

| | | Wt (gm) | | | | | | |
|---|---|---|---|---|---|---|---|---|
| Component | Source | Ex 8 | Ex 9 | Ex 10 | Ex 11 | Ex 12 | Ex 13 | Ex 14 |
| NaCl | Fisher | 6 | 6 | 5.5 | 5.5 | 5.5 | 5.5 | 6 |
| H₃BO₃ | Fisher Science ED | 0 | 0 | 0 | 0 | 0 | 0 | 4.5 |
| Na₂B₄O₇•10H₂O | Fisher Science ED | 0 | 0 | 0 | 0 | 0 | 0 | 0.25 |
| Monobasic KH₂PO₄ | Fisher | 1.44 | 1.44 | 1.44 | 1.44 | 1.98 | 1.98 | 0 |
| Disodium hydrogen | Fisher | 2.57 | 2.57 | 2.57 | 2.57 | 3.53 | 3.53 | 0 |
| phosphate dihydrate | | | | | | | | |
| CaDTPA | ISP | 0.4 | 0.4 | 0.4 | 0.4 | 0.4 | 0.4 | 0 |
| | Columbus | | | | | | | |
| Ca₂DTPA | ISP | 0 | 0 | 0 | 0 | 0 | 0 | 0.3 |
| | Columbus | | | | | | | |
| tyloxapol | Sigma | 0 | 1 | 0 | 1 | 0 | 1 | 0 |
| Trisodium citrate | Fisher | 0 | 0 | 6.5 | 65 | 0 | 0 | 0 |
| PQ-42 60% | Buchman | 0.08 | 0.08 | 0.08 | 0.08 | 0.08 | 0.08 | 0.125 |
| WSCP | Labs | | | | | | | |

The contact lens disinfection solutions from Examples 8-14 were tested for antimicrobial efficacy using the stand-alone procedure described in ISO 14729. Opti-Free Replenish (commercially available from Alcon, and containing Polyquaternium 1, (PQ-1) and myristamidopropyl dimethylamine (Aldox) as disinfecting components and a borate buffer) and AquaSoft (commercially available from AquaSoft, LLC, and containing polyaminopropyl biguanide (0.0001%) as a disinfecting component and a phosphate buffer) multipurpose solutions were also tested for comparison. Each solution was challenged with five different organisms. Bacteria used were Pseudomonas aeruginosa, Staphylococcus aureus, and Serratia marcescens. Fungi used were Candida albicans and Fusarium solani. Test organisms were cultured from representative ATCC strains as described in ISO 14729.

A ten milliliter aliquot of the test contact lens disinfection solution was placed in a sterile borosilicate glass or polypropylene screw cap test tube. To this solution was added a 0.01-0.1 milliliter aliquot of a suspension of the representative test organism in organic soil. This initial inoculum of the test organism was between 1×10⁵ and 1×10⁶ CFU/ml upon dilution with the test solution. Aliquots of the solution were taken at 25%, 50%, 75% and 100% of the minimum recommended disinfection time, MRDT of 6 hours. The residual disinfectant activity of each aliquot was neutralized and the solution plated for microbe enumeration. Log reductions for each organism were calculated for each time point tested by subtracting the remaining viable organisms from the initial inoculum. The primary criteria for microbial reduction is 3.0 log (99.9%) for the bacteria and 1.0 log (90.0%) for the fungi, within the minimum recommended disinfection time

The results are shown in Table 5, below.

TABLE 95

| | [Buffer] | [citrate] | [tyloxopol] | Log reduction @ 6 hrs | | | | |
|---|---|---|---|---|---|---|---|---|
| Ex | ppm | ppm | ppm | PA | SA | SM | CA | FS |
| 8 | P-1440 | 0 | 0 | 4.1 | 4.2 | 4.1 | 1.4 | 2.1 |
| 9 | P-1440 | 0 | 1000 | 4.1 | 4.2 | 4.1 | 1.5 | 2.5 |
| 10 | P-1440 | 650 | 0 | 4.1 | 4.2 | 4.1 | 1.4 | 1.9 |
| 11 | P-1440 | 650 | 1000 | 4.1 | 4.2 | 4.1 | 1.4 | 1.8 |
| 12 | P-1980 | 0 | 0 | 4.1 | 4.2 | 4.1 | 2.2 | 2.6 |
| 13 | P-1900 | 0 | 1000 | 4.1 | 4.2 | 4.1 | 2.2 | 2.6 |
| 14 | B | 0 | 0 | 4.7 | 4.6 | 4.4 | 0.7 | 2.7 |

| | | | | | |
|---|---|---|---|---|---|
| Opti-Free Replenish (borate buffer) | >4.1 | 4.1 | 3.0 | 2.0 | 1.7 |
| AquaSoft (phosphate buffer) | >4.1 | >4.2 | 3.8 | 0.3 | 1.5 |

PQ-42—Polyquaternium-42
PA—pseudomonas aeruginosa
SA—staph aureus
SM—serratia marcescens
CA—candida albicans
FS—fusarium solani

The solutions of the present invention containing phosphate buffer, Examples 8-13, display markedly improved efficacy against candida albicans (1.4-2.2 log reduction) compared to the borate buffered solution of Example 14 (0.7 log reduction). There was no reason to expect that changing the buffer would increase the efficacy of the disinfection capability of the solution by two to three times. Moreover, it is clear that it is not the phosphate buffer alone that is responsible for the improvement in efficacy against candida albicans as the AquaSoft solution, which also uses a phosphate buffer solution displays only a 0.3 log reduction. Examples 9-11 and 13 also show that additional components, such as tyloxapol and sodium citrate may be added to the ophthalmic compositions of the present invention without any reduction in fungal or antimicrobial efficacy.

We claim:

1. An ophthalmic composition comprising a pH between about 6 and about 8 and about 50 to about 1000 ppm hydrogen peroxide, phosphate buffer.

2. The ophthalmic composition of claim 1 wherein said phosphate buffer comprises a total phosphate concentration of about 5 to about 100 mmol.

3. The ophthalmic composition of claim 1 wherein said phosphate buffer comprises a total phosphate concentration between about 25 to about 50 mmol.

4. The ophthalmic composition of claim 1 wherein said phosphate buffer comprises from about 0.05 wt % to about 0.4 wt % monobasic phosphoric acid salt and from about 0.1 wt % to about 0.8 wt % dibasic phosphoric acid salt.

5. The ophthalmic composition of claim 1 comprising between about 0.15 to about 0.5 weight % phosphate buffer, based upon the weight of all components in the solution.

6. The composition of claim 1 wherein said hydrogen peroxide is present in a concentration between about 100 and about 500 ppm.

7. The composition of claim 1 wherein said hydrogen peroxide is present in a concentration between about 100 and about 300 ppm.

8. The composition of claim 1 wherein said pH is between about 6.5 about 7.5.

9. The composition of claim 1 further comprising about 100 ppm to about 2000 ppm of at least one chlorite compound.

10. The composition of claim 1 or 9 further comprising about 10 to 100 ppm of at least one saturated, polymeric quaternium salt.

11. The composition of claim 1 further comprising at least one stabilizer.

12. The composition of claim 11 wherein said at least one stabilizer is selected from the group consisting of diethylenetriamine pentaacetic acid salt, selected from the group consisting monocalcium salt or zinc salt of diethylenetriamine pentaacetic acid and mixtures thereof.

13. The solution of claim 11 wherein said diethylenetriamine pentaacetic acid salt is present in a concentration between about 50 and about 1,500 ppm.

14. The solution of claim 11 wherein said diethylenetriamine pentaacetic acid salt is present in a concentration between about 100 and about 1,000 ppm.

15. The composition of claim 1 further comprising water.

16. The composition of claim 1 or 12, further comprising at least one stabilizer comprising diethylenetriamine pentamethylenephosphonic acid salt.

17. The composition of claim 16 wherein said diethylenetriamine pentamethylenephosphonic acid salt is present in a concentration up to about 1000 ppm.

18. The composition of claim 16 wherein said diethylenetriamine pentamethylenephosphonic acid salt is present in a concentration between about 100 ppm to about 1000 ppm.

19. The composition of claim 11 comprising at least two stabilizers.

20. The composition of claim 1 further comprising at least one additional component selected from the group consisting of tonicity adjusting agents, active agents, lubricating agents, disinfecting agents, surfactants and mixtures thereof.

21. The composition of claim 10 wherein said at least one saturated, polymeric quaternium salt comprises poly[oxyethylene(-dimethylimino)ethylene-(dimethylimino)ehthylene dichloride.

22. The composition of claim 10 wherein said at least one chlorite compound is present in an amount of about 100 ppm to about 1000 ppm.

23. The composition of claim 22 wherein said chlorite compound is selected from the group consisting of water soluble alkali metal chlorites, water soluble alkaline metal chlorites and mixtures thereof.

24. The composition of claim 22 wherein said chlorite compound is selected from the group consisting of potassium chlorite, sodium chlorite, calcium chlorite, magnesium chlorite and mixtures thereof.

25. The composition of claim 22 wherein said chlorite compound comprises sodium chlorite.

26. The composition of claim 22 wherein said chlorite compound is present in an amount between about 100 and about 500 ppm.

27. The composition of claim 25 wherein said chlorite compound is present in an amount between about 200 and about 500 ppm.

28. The composition of claim 1 further comprising about 0.1 to about 1 weight % of at least one lubricating agent.

29. The composition of claim 1 wherein said composition is an ophthalmic solution.

30. The composition of claim 28 wherein said lubricating agent comprises polyvinyl pyrrolidone.

31. The composition of claim 1 further comprising at least one disinfection enhancer.

32. The composition of claim 31 wherein said at least one disinfection enhancer is selected from the group consisting of C₅₋₂₀ polyols.

33. The composition of claim 31 wherein said at least one disinfection enhancer is present in an amount between about 50 ppm and about 2000 ppm and is selected from the group consisting of 1,2-octanediol, glycerol monocaprylate, sorbitan monolaurate (TWEEN 80) and mixtures thereof.

34. The composition of claim 31 wherein said disinfection enhancer comprises tyloxapol.

35. The ophthalmic composition of claim 1 further comprising at least one chlorite compound, at least one saturated, polymeric quaternium salt, PVP, at least one diethylenetriamine pentaacetic acid salt and at least one surfactant.

36. The ophthalmic composition of claim 1 further comprising sodium citrate, tyloxapol, poloxamine type surfactant and at least one disinfection enhancer.
